(19) United States
(12) Patent Application Publication
(10) Pub. No.: US 2026/0317779 A1
(43) Pub. Date: Oct. 15, 2026

(54) REMOTE-CONTROLLED ALTERATION/INDUCTION OF CELLULAR APOPTOSIS AND METHODS OF USE

(71) Applicants: Neemat G. Frem, Keserwan (LB); Nour N. Frem, Keserwan (LB); Assaad A. Eid, Paris (FR)

(72) Inventors: Neemat G. Frem, Keserwan (LB); Nour N. Frem, Keserwan (LB); Assaad A. Eid, Paris (FR)

(21) Appl. No.: 19/466,756

(22) Filed: Feb. 2, 2026

Related U.S. Application Data
(63) Continuation of application No. PCT/IB2024/058288, filed on Aug. 26, 2024.
(60) Provisional application No. 63/578,710, filed on Aug. 25, 2023.

(51) Int. Cl. C12N 15/86; A61K 31/7105; A61K 48/00; A61K 51/04; A61P 35/00

(52) U.S. Cl. CPC C12N 15/86; A61K 31/7105; A61K 48/005; A61K 51/0491; A61P 35/00; C12N 2740/15032; C12N 2800/60; C12N 2840/007

(57) ABSTRACT

Provided herein are systems, methods, and compositions for a Remote-Controlled Alteration/Induction of Cellular Apoptosis. In one embodiment, the method comprises radio-controlled mRNA therapy to treat cancer.

### CROSS-REFERENCE TO RELATED APPLICATIONS

The present application claims priority to PCT application serial no. PCT/IB2024/058288, filed Aug. 26, 2024, which claims priority to U.S. provisional application Ser. No. 63/578,710, filed Aug. 25, 2023, each herein incorporated by reference in its entirety.

### BACKGROUND

The invention generally relates to the treatment of a diseased state, and more particularly, to the treatment of cancer.

Cancer is a disease characterized by uncontrolled in vivo cell division and growth. In the United States, about one third of all women and half of all men will experience cancer during their lifetime. Gene therapy for manipulating disease-related peptides and their functions provides a more targeted approach to disease diagnosis, treatment, and management due to the adverse consequences of standard therapies such as chemotherapy and radiation therapy currently in use. However, gene therapy presents several challenges, including undesirable immune responses and safety issues due to the incorporation of genes at random locations within the genome. Thus, there is a need for improved therapeutic methods for treating tumors.

In the current state of the art, cell death alteration using radiotherapy has several drawbacks since the radiation sources are used with relatively high power and cannot have high precision in cell targeting. While chemotherapy in its current state, is associated with many off-target effects due to the cancer/cell cancer environment interactions that lead to an alteration in the immune system resulting in a decrease in treatment efficiency. Furthermore, the major challenge in the current immunotherapies and genetic personalized medicine, is to strike the adequate homeostatic balance between the lethality of the treatment and its stealth capability to evade the immune system.

The present invention attempts to solve these problems, as well as others.

### SUMMARY OF THE INVENTION

Provided herein are systems, methods, and compositions for a Remote-Controlled Alteration/Induction of Cellular Apoptosis. In one embodiment, method the treatment comprises targeting undesired cells with an mRNA sequence through the circulatory system or locally without stimulating immune response; remotely activating the mRNA sequence to activate a lethal mode of the undesired cells. In one embodiment, the method for remotely activating the mRNA sequence is from an electromagnetic source that covers a specific body location or systemically, with no side effects on normal surrounding tissue of the subject.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIGS. 1A-1D are images showing in vitro results: Cell death induction remote-controlled constructed mRNA, FIG. 1A: Human cancer epithelial cells before transfection; FIG. 1B: Human cancer epithelial cells 6 hours; after transfection, FIG. 1C: Human cancer epithelial cells 24 hours after transfection, FIG. 1D: shows Human cancer epithelial cells 48 hours after transfection.

FIGS. 2A-2D are photographs showing the rectal change and bleeding associated with CRC development were reversed using remote controlled constructed mRNA; FIG. 2A shows CRC-Induced Rectal Prolapse; FIG. 2B shows CRC-Induced Rectal Bleeding; FIG. 2C shows CRC-Induced Diarrhea; and FIG. 2D shows CRC mice after radio-controlled mRNA treatment.

FIGS. 3A-3D are microscope images of H&E staining for the Colorectal Cancer Experiments. FIG. 3A is the control mice; FIG. 3B is the CRC mice; FIG. 3C is the CRC mice treated with a combination of Radiotherapy and Chemotherapy; and FIG. 3D is the CRC mice treated with the radio-controlled mRNA.

FIGS. 4A-4D: are microscope images of PAS staining Colorectal Cancer Experiments. FIG. 4A is the control mice; FIG. 4B is the CRC mice; FIG. 4C is the CRC mice treated with a combination of Radiotherapy and Chemotherapy; and FIG. 4D is the CRC mice treated with the radio-controlled mRNA.

FIGS. 5A-5D are microscope images of Masson's trichrome staining for the Colorectal Cancer Experiments. FIG. 5A is the control mice; FIG. 5B is the CRC mice; FIG. 5C is the CRC mice treated with a combination of Radiotherapy and Chemotherapy; and FIG. 5D is the CRC mice treated with the radio-controlled mRNA.

FIG. 6: is a graph of the Survival Curve (Kaplan-Meier) for the Colorectal Cancer Experiments.

FIG. 7: is a graph of the Survival Curve (Kaplan-Meier) for the Breast Cancer Experiments.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes, or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.,” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

Nanoparticle composition: as used herein, a “nanoparticle composition” is a composition that comprises one or more lipids. Nanoparticle compositions are typically approximately microns or less in size and may include lipid bilayers. Nanoparticle compositions encompass Lipid Nanoparticles (LNPs), liposomes (e.g., lipid vesicles), and lipid complexes. For example, the nanoparticle composition may be a liposome having a lipid bilayer with a diameter of 500 nm or less.

The terms “promoter” and “expression control sequence” are used herein to refer to a nucleic acid control sequence that directs transcription of a nucleic acid. Promoter sequences are typically near the start site of transcription, such as a TATA element in the case of a polymerase II type promoter. A promoter can also include distal enhancer or repressor elements, which can be located as much as several thousand base pairs from the start site of transcription. Promoters include constitutive and inducible promoters. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

Expression vectors for use as described herein can include virally-derived vectors, e.g., recombinant adeno-associated virus (AAV) vectors, retroviral vectors, adenoviral vectors, modified vaccinia Ankara (MVA) vectors, and lentiviral (e.g., HSV-1-derived) vectors (see, e.g., Brouard et al. (2009) British J. Pharm. 157:153). Virally-derived vectors for therapeutic use are typically rendered replication incompetent or attenuated. For example, in the case of an adenoviral vector, the adenoviral genome can be modified to remove the E1 and E3 genes. For production, the replication deficient vector can be administered to a cell that expresses the E1 gene such that recombinant adenovirus (rAd) is produced by the cell. This rAd can be harvested and used for a single round of infection to deliver the transgenic composition to another cell within a mammal in order to elicit immune responses to an encoded polypeptide antigen.

Examples of suitable viral vectors include adenovirus 5, including, for example, Ad5 with deletions of the E1/E3 regions and Ad5 with a deletion of the E4 region as described in U.S. Pat. No. 8,222,224 and Scallan et al. Clinical and Vaccine Immunology 2013; 20 (1): 85-94. An exemplary Ad5 viral vector backbone is provided in SEQ ID NO: 7. Other suitable adenoviral vectors include strains 2, orally tested strains 4 and 7, enteric adenoviruses 40 and 41, and other strains (e.g. Ad34, Ad26, or Ad35) that are sufficient for delivering an antigen and eliciting an adaptive immune response to the transgene antigen [Lubeck et al., Proc Natl Acad Sci USA, 86 (17), 6763-6767 (1989); Shen et al., J Virol, 75 (9), 4297-4307 (2001); Bailey et al., Virology, 202 (2), 695-706 (1994)]. The viral vector does not need to have been isolated from humans but can come from a non-human such as chimpanzee adenovirus 3 (ChAd3) (see, e.g., Colloca et al. (2012) Sci. Transl. Med. 4:115; Stanley et al. (2014) Nat. Med. doi:10.1038/nm.3702). In some embodiments, the adenoviral vector is a live, replication incompetent adenoviral vector (such as E1 and E3 deleted rAd5), live and attenuated adenoviral vector (such as the E1B55K deletion viruses), or a live adenoviral vector with wild-type replication.

Transcriptional and translational control sequences in expression vectors to be used as described herein can be provided by viral sources. For example, commonly used promoters and enhancers are derived, e.g., from beta actin, adenovirus, simian virus (SV40), and human cytomegalovirus (CMV). For example, vectors allowing expression of proteins under the direction of the CMV promoter, SV40 early promoter, SV40 later promoter, metallothionein promoter, murine mammary tumor virus promoter, Rous sarcoma virus promoter, transducer promoter, or other promoters shown effective for expression in mammalian cells are suitable. Additional viral and non-viral promoter, control and/or signal sequences may be used, provided such control sequences are compatible with the host cells to be transfected.

mRNA is a synthetic mRNA. In some aspects, the synthetic mRNA comprises at least one unnatural nucleobase. In some aspects, all nucleobases of a certain class have been replaced with a non-natural nucleobase (e.g., all uridine in the polynucleotides disclosed herein can be replaced with a non-natural nucleobase, such as 5-methoxyuridine). In some aspects, the polynucleotide (e.g., synthetic RNA or synthetic DNA) comprises only natural nucleobases, i.e., A, C, T and U in the case of synthetic DNA, or A, C, T and U in the case of synthetic RNA.

Immune response: The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

Methods of Administration: As used herein, “methods of administration” may include intravenous, intramuscular, intradermal, subcutaneous, or other methods of delivering a composition to a subject. A method of administration may be selected to target delivery (e.g., to specifically deliver) to a specific region or system of a body.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A, C, T and U in the case of a synthetic DNA, or A, C, T, and U in the case of a synthetic RNA.

Nucleic acid sequence: The terms “nucleic acid sequence,” “nucleotide sequence,” or “polynucleotide” are used interchangeably and refer to a contiguous nucleic acid sequence. The sequence can be either single stranded or double stranded DNA or RNA, e.g., an mRNA.

The phrase “nucleotide sequence encoding” and variants thereof refers to the nucleic acid (e.g., an mRNA or DNA molecule) coding sequence that comprises a nucleotide sequence which encodes a polypeptide or functional fragment thereof as set forth herein. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of a cancer treatment.

Targeted cells: As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient.

Target tissue or undesired cells: As used herein “target tissue” or “undesired cells” refers to any one or more tissue types or cell types of interest in which the delivery of a polynucleotide would result in a desired biological and/or pharmacological effect. Examples of target tissues of interest include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

Transcription: As used herein, the term “transcription” refers to methods to introduce exogenous nucleic acids into a cell. Methods of transfection include, but are not limited to, chemical methods, physical treatments and cationic lipids or mixtures.

Transfection: As used herein, “transfection” refers to the introduction of a polynucleotide into a cell wherein a polypeptide encoded by the polynucleotide is expressed (e.g., mRNA) or the polypeptide modulates a cellular function (e.g., siRNA, miRNA). As used herein, “expression” of a nucleic acid sequence refers to translation of a polynucleotide (e.g., an mRNA) into a polypeptide or protein and/or post-translational modification of a polypeptide or protein.

Treating, treatment, therapy: As used herein, the term “treating” or “treatment” or “therapy” refers to partially or completely alleviating, ameliorating, improving, relieving, delaying onset of, inhibiting progression of, reducing severity of, and/or reducing incidence of one or more symptoms or features of a hyper-proliferative disease, e.g., cancer. For example, “treating” cancer can refer to inhibiting survival, growth, and/or spread of a tumor. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

### DESCRIPTION OF EMBODIMENTS

A novel process to irradicate specific cells by a targeting method comprising administering specific mRNA sequence using vectors or Lipid nanoparticles (LNPs) that alter/activate the apoptotic process of the target cells (P53, Casp 3, Casp 9, etc.) upon specific stimulation by radio-control external factors. In one embodiment, the mRNA sequence is synthetic mRNA incorporated in lentiviral vector or LNPs.

The mRNA sequence is created using in vitro transcription and the construct comprises: a Radio-control inducible promotor Accession numbers: HQ542862, HQ418223, HQ418224, EF536082 (wherein the promotor comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: HQ542862) that enables the apoptotic genes to be altered/activated; and a specific and personalized coding sequence that match the target gene sequence in specific cells that are altered in several pathophysiological disease such as cancer (BRACA1, APC, or others), or Atherosclerosis (APOE, etc.), or diabetes (GCK, TCF7L2, ABCC8, etc.), and fatty liver disease and cirrhosis, and other diseases.

The target tissues/cells to be treated can be tissue specific, cell specific or a combination of both, and can be geofenced for a specific body localization treatment, or systemic for a body wide treatment.

In specific clinical cases the target can be a whole organ (i.e., prostate, and others).

The synthetic mRNA may be administrated by Local injection or Systemically through an IV intervention.

In some embodiments, administration of the mRNA sequence, e.g., according to the methods disclosed above, treats a cancer. In some embodiments, the cancer is selected from the group consisting of adrenal cortical cancer, advanced cancer, anal cancer, aplastic anemia, bile duct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, hepatocellular carcinoma (HCC), non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor, secondary cancers caused by cancer treatment, and any combination thereof.

In one embodiment, the method is provided as a cancer treatment and, due to the fact that this methodology alters the apoptotic process in undesired cells or microorganisms, with a homing mechanism to allow cell specific targeting, and the construct is built to be remotely activated by specific electromagnetic sources, the method can be applied in the future to improve organs functions by eradicating certain kind of cells that affect the good performance of organs (ex-fat in liver, . . . ). In another embodiment, the method of treatment can also be used to treat bacterial or virus infection as a specific bacterial or virus killer if the targeting mechanism is directed toward specific bacteria or viruses' proteins. In other embodiments, the method replaces surgery, to ablate cysts or small organs (ex-prostate, breast tumors, and the like).

In one embodiment, the method the treatment is finely designed to target the undesired cells through the circulatory system or locally without stimulating immune response because of its non-combating mode while enroute to the specific targeted cells. Once properly attached to its the targeted undesired cells, the mRNA sequence will be remotely activated to revert to a lethal mode. Furthermore, in one embodiment, the electromagnetic source is used to cover a specific body location or systemically, with very low power of less than 10 gray (Gy) and hence no side effects on normal surrounding.

Gray is the unit of ionizing radiation dose in the International System of Units (SI), defined as the absorption of one joule of radiation energy per kilogram of matter. It is used as a unit of the radiation quantity absorbed dose that measures the energy deposited by ionizing radiation in a unit mass of matter being irradiated and is used for measuring the delivered dose in radiotherapy, food irradiation and radiation sterilization. It is important in predicting likely acute health effects, such as acute radiation syndrome and is used to calculate equivalent dose using the sievert, which is a measure of the stochastic health effect on the human body. The average radiation dose from an abdominal X-ray is 0.7 millisieverts (0.0007 Sv), that from an abdominal CT scan is 8 mSv, that from a pelvic CT scan is 6 mGy, and that from a selective CT scan of the abdomen and the pelvis is 14 mGy.

### Nucleotide Sequences

In some embodiments, the mRNA sequence comprises a nucleotide sequence (e.g., an ORF) encoding a P53 polypeptide, e.g., a P53 polypeptide comprising the EC domain of P53 or a functional portion thereof, wherein the P53 polypeptide comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: AAD28535.

In some embodiments, the mRNA sequence comprises a nucleotide sequence (e.g., an ORF) encoding a Caspase 9 polypeptide, e.g., a Caspase 9 polypeptide comprising the EC domain of Caspase 9 or a functional portion thereof, wherein the Caspase 9 polypeptide comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: ACV31396.1.

In some embodiments, the mRNA sequence comprises a nucleotide sequence (e.g., an ORF) encoding a Caspase 3 polypeptide, e.g., a Caspase 3 polypeptide comprising the EC domain of Caspase 3 or a functional portion thereof, wherein the Caspase 3 polypeptide comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: KAF6460065.1.

In some embodiments, the mRNA sequence comprises a nucleotide sequence (e.g., an ORF) encoding a BRACA1 polypeptide, e.g., a BRACA1 polypeptide comprising the EC domain of BRACA1 or a functional portion thereof, wherein the BRACA1 polypeptide comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: KAG2460542.1.

In some embodiments, the mRNA sequence comprises a nucleotide sequence (e.g., an ORF) encoding an APC polypeptide, e.g., an APC polypeptide comprising the EC domain of APC or a functional portion thereof, wherein the APC polypeptide comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: AAA03586.1.

In some embodiments, the mRNA sequence comprises a nucleotide sequence (e.g., an ORF) encoding an APOE polypeptide, e.g., an APOE polypeptide comprising the EC domain of APoE or a functional portion thereof, wherein the APOE polypeptide comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: EAX06743.1.

In some embodiments, the mRNA sequence comprises a nucleotide sequence (e.g., an ORF) encoding a GCK polypeptide, e.g., a GCK polypeptide comprising the EC domain of GCK or a functional portion thereof, wherein the GCK polypeptide comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: NP 001294919.1.

In some embodiments, the mRNA sequence comprises a nucleotide sequence (e.g., an ORF) encoding a TCF7L2 polypeptide, e.g., a TCF7L2 polypeptide comprising the EC domain of TCF7L2 or a functional portion thereof, wherein the TCF7L2 polypeptide comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: AAI49367.1.

In some embodiments, the mRNA sequence comprises a nucleotide sequence (e.g., an ORF) encoding a ABCC8 polypeptide, e.g., a ABCC8 polypeptide comprising the EC domain of ABCC8 or a functional portion thereof, wherein the ABCC8 polypeptide comprises an amino acid sequence at least 70%, at least 75%, at least 80%, at least 85%, at least 90%, at least 95%, at least 96%, at least 97%, at least 98%, at least 99%, or 100% identical to amino acids SEQ ID NO: XP_015719625.1.

The below examples obtained using the novel technique of remote-controlled alteration/induction of cellular apoptosis.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: In Vitro Results: Cell Death Induction Using Remote-Controlled mRNA

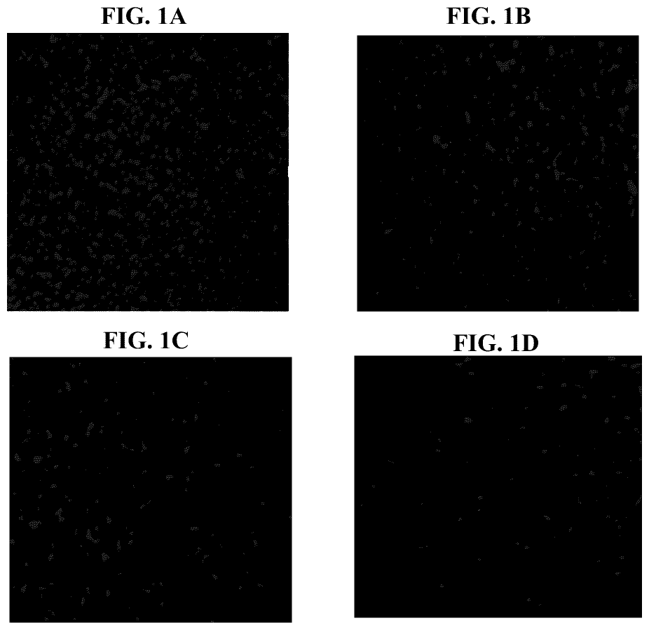

FIGS. 1A-1D show cultured human cancer epithelial cells were subjected to transfection using lentiviral vectors carrying a radio-controlled promoter that triggers the activation of apoptotic genes. Furthermore, the lentiviral vectors contained a specifically designed sequence to target and recognize the colon cancer gene biomarker APC. The experiment was repeated four times (n=4), and the results consistently exhibited a significant reduction in cancer cell population subsequent to transfection.

### Example 2

Cultured human cancer epithelial cells (CaCo2) were subjected to transfection using mRNA carrying a radio-controlled promoter that triggers the activation of apoptotic genes. The experiment was repeated four times (n=4), and the results consistently exhibited an increase in apoptosis using 2 different techniques of measurement the Annexin V binding assay and the cellular DNA fragmentation Assay. Results are expressed as percentage of apoptosis.

Table 1 showing in vitro results where the cells death induction after transfection with a radio-controlled constructed mRNA. The experiments were performed using 2 techniques the Annexin V binding Assay and the Cellular DNA fragmentation Assay. Both gives the percentage of apoptotic cells.

TABLE 1
Cell death induction using remote-controlled constructed mRNA cancer

| | Human cancer | Human cancer | Human cancer | Human cancer |
|---|---|---|---|---|
| | epithelial cells | epithelial cells | epithelial cells | epithelial cells |
| | before | 6 hours after | 24 hours after | 48 hours after |
| Assays | transfection | transfection | transfection | transfection |
| Annexin V | 3 ± 0.07 | 23.6 ± 3.4 | 43.9 ± 6.4 | 68.9 ± 8.2 |
| Binding Assay (% | | | | |
| of apoptotic cells) | | | | |
| Cellular DNA | 5 ± .12 | 30.4 ± 5.7 | 48.6 ± 8.2 | 72.8 ± 8.2 |
| Fragmentation (% | | | | |
| of apoptotoic | | | | |
| cells) | | | | |

### Example 3-Animal Experiments Results 1: Cell Death Induction Using Remote-Controlled Constructed mRNA

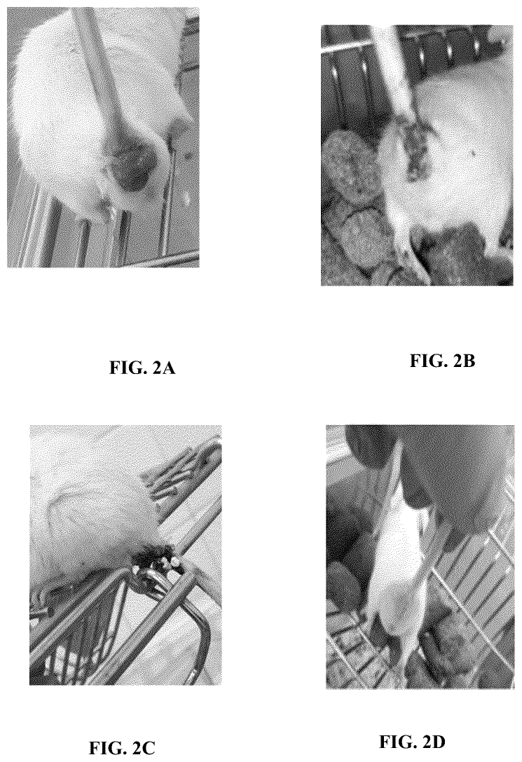

Mice that harbor mutant APC alleles, mimicking human colon cancer pathogenesis, were used to intensify the development of colorectal cancer, these mice were subsequently treated with azoxymethane/DSS to exacerbate cancer development. This experimental group was designated as CRC mice. Following an 8-week period from the azoxymethane/DSS protocol, the mice were subjected to the synthesized mRNA, delivered via lentivirus, alter which the radio-controlled promoter was activated, the mice were then monitored for a duration of 3 months before being sacrificed (n=4). n parallel experiments, a control group consisting of non-CRC mice (n=4) was utilized, along with a group of CRC mice that were not subjected to any treatment (n=4). The data show a significant decrease in polyp number and size upon treatment (·p<0.05), Furthermore, phenotypic rectal change and bleeding were reversed upon treatment (FIG. 2A-2C).

Animal Experiments Results 2: Cell Death Induction Using Remote-Controlled Constructed mRNA

TABLE 2a

| Animal model | Number of polyps | Polyp size (mm³) |
|---|---|---|
| Control mice (n = 4)1 | Non-Observed | Non-Observed |
| CRC mice (n = 4)1 | 15 +/− 4 | 4 +/− 0.8 |
| CRC mice treated with the | 6 +/− 2* | 1 +/− 0.2* |
| designed mRNA (n = 4) | | |

Mice that harbor mutant APC alleles, mimicking h man colon cancer pathogenesis, were used to intensify the development of colorectal cancer, these mice were subsequently treated with azoxymethane/DSS to exacerbate cancer development. This experimental group was designated as CFIC mice. Following an 8-week period from the azoxymethane/DSS protocol, the mice were subjected to the synthesized mRNA, delivered via lentivirus, alter which the radio-controlled promoter was activated. The mice were then monitored for a duration of 3 months before being sacrificed (n=4). n parallel experiments, a control group consisting of non-CRC mice (n=4) was utilize-0, along with a group of CRC mice that were not subjected to any treatment (n=4), This data show a significant decrease in polyp number and size upon treatment (*p<0.05) (Table 2a). Furthermore, rectal change and bleeding associated with CRC development were reversed mRNA treatment (FIGS. 2A-2C).

Cell Death Induction Using Remote-Controlled mRNA:

Cultured human cancer epithelial cells (CaCo2) were subjected to transfection using mRNA carrying a radio-controlled promoter that triggers the activation of apoptotic genes. The experiment was repeated four times (n=4), and the results consistently exhibited an increase in apoptosis using 2 different techniques of measurement the Annexin V binding assay and the cellular DNA fragmentation Assay. Results are expressed as percentage of apoptosis. Table 1b showing in vitro results where the cells death induction after transfection with a radio-controlled constructed mRNA. The experiments were performed using 2 techniques the Annexin V binding Assay and the Cellular DNA fragmentation Assay. Both gives the percentage of apoptotic cells.

TABLE 1b
Cultured human cancer epithelial cells (CaCo2) were subjected to
transfection using lentiviral vectors carrying a radio-controlled
promoter that triggers the activation of apoptotic genes.

| | Human cancer | Human cancer | Human cancer | Human cancer |
|---|---|---|---|---|
| | epithelial cells | epithelial cells | epithelial cells | epithelial cells |
| | before | 6 hours after | 24 hours after | 48 hours after |
| Assays | transfection | transfection | transfection | transfection |
| Annexin V | 3 ± 0.07 | 23.6 ± 3.4* | 43.9 ± 6.4* | 68.9 ± 8.2* |
| Binding Assay | | | | |
| (% apoptotic | | | | |
| cells) | | | | |
| Cellular DNA | 5 ± 1.2 | 30.4 ± 5.7* | 48.6 ± 8.2* | 72.8 ± 8.2* |
| Fragmentation | | | | |
| (% of | | | | |
| apoptotoic | | | | |
| cells) | | | | |

*P<0.05 vs. Before Transfection

FIGS. 1A-1D show cultured human cancer epithelial cells subjected to transfection using lentiviral vectors carrying a radio-controlled promoter that triggers the activation of apoptotic genes. Furthermore, the lentiviral vectors contained a specifically designed sequence to target and recognize the colon cancer gene biomarker APC. The experiment was repeated four times (n=4), and the results consistently exhibited a significant reduction in cancer cell population subsequent to transfection.

Table 2b shows human breast cancer cell line with estrogen, progesterone and glucocorticoid receptors MCF-7 that are transfected using mRNA carrying a radio-controlled promoter that triggers the activation of apoptosis. The MCF-7 cell line is estrogen receptor (ER) positive, progesterone receptor positive, and HER2 negative, which are features commonly seen in luminal subtype breast cancers. The experiments were repeated four times (n=4), and the results consistently exhibited an increase in apoptosis subsequent to transfection using 2 different techniques of measurements the Annexin V binding assay and the cellular DNA fragmentation Assay. Results are expressed as percentage of Apoptosis (Table 2b).

TABLE 2b
Human breast cancer cell line with estrogen, progesterone and glucocorticoid
receptors MCF-7 that are transfected using mRNA carrying a radio-controlled
promoter that triggers the activation of apoptosis

| | MCF-7 cells | MCF-7 cells 6 | MCF-7 cells | MCF-7 cells 48 |
|---|---|---|---|---|
| | before | hours after | 24 hours after | hours after |
| Assays | transfection | transfection | transfection | transfection |
| Annexin V | 4.1 ± 0.13 | 27.9 ± 4.2* | 47.5 ± 7.8* | 87.5 ± 7.1* |
| Binding | | | | |
| Assay (% | | | | |
| of apoptotic | | | | |
| cells) | | | | |
| Cellular | 4.2 ± 0.87 | 42.1 ± 7.3* | 62.6 ± 10.9* | 97.5 ± 12.8* |
| DNA | | | | |
| Fragmentation | | | | |
| (% of | | | | |
| apoptotoic | | | | |
| cells) | | | | |

*P<0.05 vs. Before Transfection

### Animal Experiments: Colorectal Cancer

Cell Death Induction Using Remote-Controlled Constructed mRNA

Mice harboring mutant APC alleles, mimicking human colon cancer pathogenesis, were used to intensify the development of colorectal cancer. These mice were subsequently treated with azoxymethane/DSS to exacerbate cancer development, designating this experimental group as CRC mice. Following an 8-week period from the azoxymethane/DSS protocol, the mice were subjected to synthesized mRNA, delivered via lentivirus, after which a radio-controlled promoter was activated. The mice were then monitored for a duration of 32 weeks before being sacrificed (n=10).

In parallel experiments, a control group consisting of non-CRC mice (n=10) was utilized, along with a group of CRC mice that were not subjected to any treatment (n=10), and a group (n=10) that was subjected to a combination of radiotherapy (External Beam Radiotherapy to the pelvis of 45-50.4 Gy in 25-28 fractions over 6 weeks) and chemotherapy (continuous infusion of 5-FU during radiotherapy) and followed for the same duration.

This data shows that both radiotherapy and chemotherapy treatments reversed rectal prolapse, rectal bleeding and stopped diarrhea, as shown in FIGS. 2A-2D. Besides, a significant decrease in polyp number and size upon treatment is obtained with either the radio-controlled mRNA therapy or the combination of radiotherapy and chemotherapy (*p<0.05 vs CRC), as shown in Table 3.

TABLE 3
Polyps number and size; Colorectal Cancer Experiments

| Animal model | Number of polyps | Polyp size (mm³) |
|---|---|---|
| Control mice (n = 10) | Non-Observed | Non-Observed |
| CRC mice (n = 10) | 17 ± 4 | 5 ± 1.2 |
| CRC mice treated with a combination of | 10 ± 3* | 2 ± 0.5* |
| Radiotherapy and Chemotherapy (n = 10) | | |
| CRC mice treated with the radio-controlled | 4 ± 1*^{#} | 0.4 ± 0.08*^{#} |
| mRNA (n = 10) | | |

*p<0.05 vs. Control; #p<0.05 vs. Treatment with a Combination of Radiotherapy and Chemotherapy

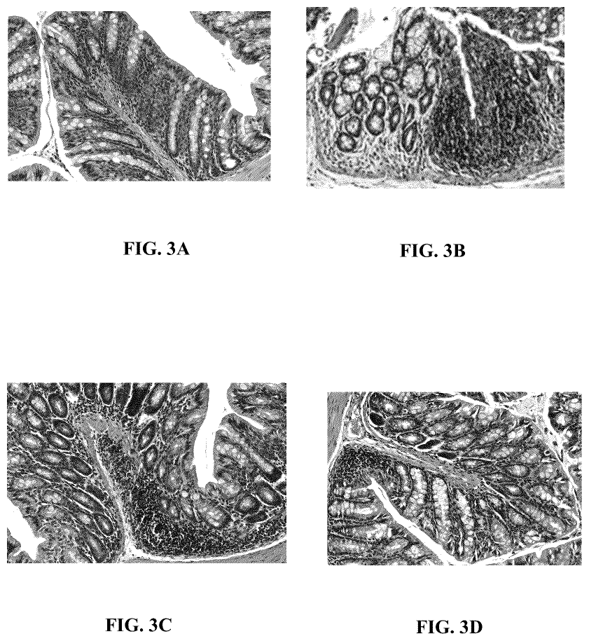

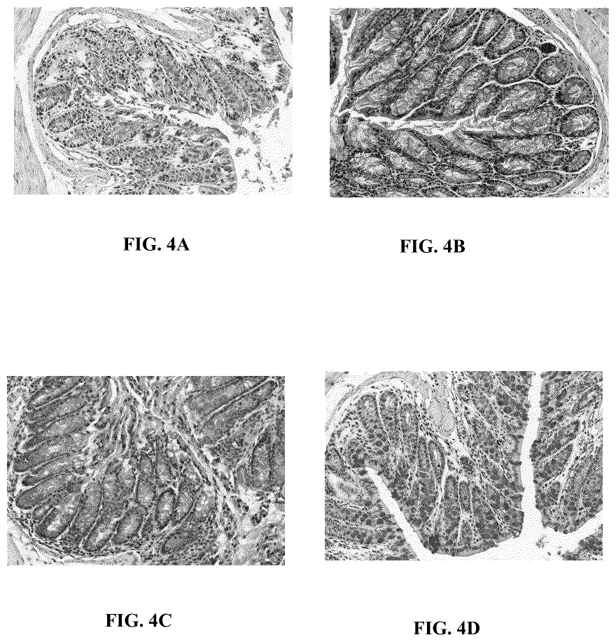

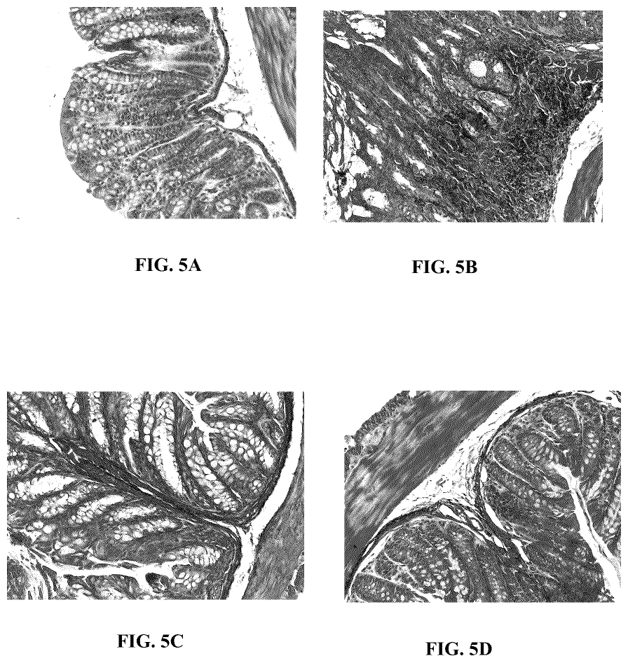

Furthermore, the colons were histologically examined to determine the degree of colon injury (FIGS. 3-5). These findings indicate a loss of crypts and brush border along with moderate to severe inflammation in CRC mice compared to control non-CRC mice, which had healthy crypts, intact brush borders, and no inflammation (FIGS. 3A, 3B). CRC was associated with a significantly higher fibrotic area, indicating increased collagen deposition in CRC colons compared to control mice (Table 4; FIGS. 4A, 4B) The sclerotic index was significantly increased in CRC mice compared to control non-CRC mice, indicating increased glycogen deposition (Table 4; FIGS. 5A, 5B).

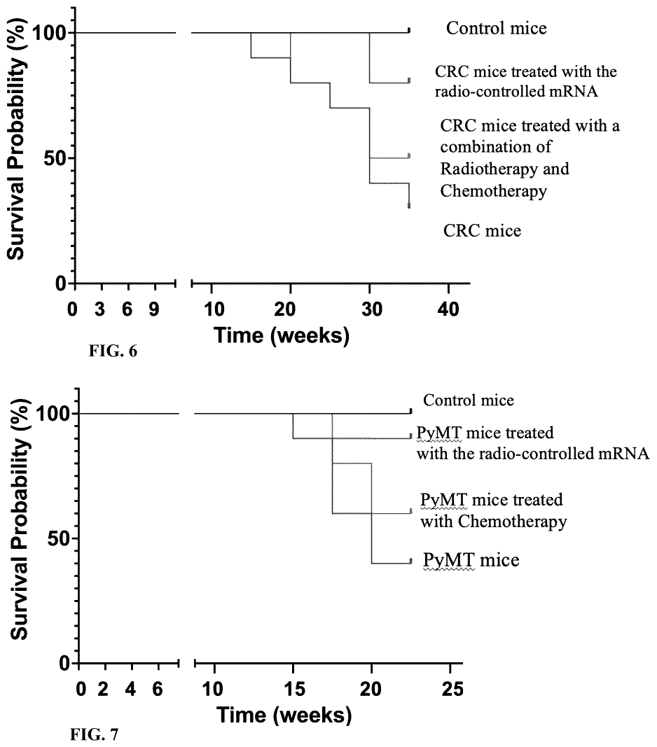

A significant improvement in the histopathological changes was observed in the treated mice, either with radio-controlled mRNA or with the combination of chemotherapy and radiotherapy (Table 4; FIGS. 3-5). Interestingly, this data shows that the mice on radio-controlled mRNA treatment had healthier colons resembling those of the control non-CRC mice, with a significant reduction in the number and size of polyps in the CRC mice treated with the radio-controlled mRNA compared to those treated with the combination of radiotherapy and chemotherapy (Table 3; Table 4; and FIGS. 3C, 3D, 4C, 4D, 5C, and 5D). Additionally, the mice treated with the combination of radiotherapy and chemotherapy continued to show mild infiltration in the mucosa and submucosa, histopathological changes that were no longer observed in the mice treated with the radio-controlled mRNA (FIG. 3). Of particular interest, the life expectancy of the mice differed significantly when comparing the different treatments. Mice on radio-controlled mRNA treatment had a higher life expectancy compared to those on the combined chemotherapy and radiotherapy treatment (FIG. 6).

TABLE 4
Fibrosis and Sclerosis; Colorectal Cancer Experiments

| | Fibrosis | Sclerotic Index |
|---|---|---|
| Animal model | Area (%) | (Fold Increase) |
| Control mice (n = 10) | 4 ± 0.5 | 1 |
| CRC mice (n = 10) | 32 ± 8.2* | 3.8 ± 1.1* |
| CRC mice treated with a | 18 ± 2.8*^{$} | 2.4 ± 0.7*^{$} |
| combination of Radiotherapy and | | |
| Chemotherapy (n = 10) | | |
| CRC mice treated with the radio- | 7.9 ± 2.1*^{#} | 1.6 ± 0.3*^{#} |
| controlled mRNA (n = 10) | | |

*p<0.05 vs. Control; $p<0.05 vs. CRC; #p<0.05 vs. Treatment with a Combination of Radiotherapy and Chemotherapy

### Animal Experiments: Breast Cancer

Cell Death Induction Using Remote-Controlled Constructed mRNA

The MMTV-PyMT transgenic mouse model that carries the polyoma virus middle T-antigen under the control of the mouse mammary tumor virus (MMTV) promoter was used in this series of experiments. The PyMT is involved in multiple oncogenic pathways that lead to an aggressive tumor phenotype such as Src, Ras, and PI3K. MMTV-PyMT females develop multifocal, poorly differentiated, highly invasive ductal carcinoma as early as 4 weeks of age, reaching the maximum tumor burden at 12 weeks of age. The PyMT mice were subjected to synthesized mRNA, delivered via lentivirus at 5 weeks of age after which a radio-controlled promoter was activated and followed up for 20 weeks (n=10).

In parallel experiments, a control group consisting of control FVB mice (n=10) was utilized, along with a group of PyMT mice that were not subjected to any treatment (n=10), and a group (n=10) that was subjected to chemotherapy (3 cycles of 20 mg/kg of Docetaxel every 2 weeks initiated at 4 weeks of age and a follow up for the same duration after treatment).

This data shows a significant decrease in tumor size upon treatment with either the radio-controlled mRNA therapy or chemotherapy (*p<0.05 vs PyMT; Table 5). Of high interest the tumor size was significantly lower in the radio-controlled mRNA mice when compared to the mice only receiving chemotherapy (#p<0.05 vs Chemotherapy; Table 5). Furthermore, and of particular interest, the life expectancy of the mice differed significantly when comparing the different treatments. Mice on radio-controlled mRNA treatment had a higher life expectancy compared to those on the chemotherapy treatment (FIG. 7).

TABLE 5
Tumor size and weight; Breast Cancer Experiments

| Animal model | Tumor size (mm³) | Tumor weight (g) |
|---|---|---|
| Control mice (n = 10) | Non-Observed | — |
| PyMT mice (n = 10) | 640 ± 65.6 | 1.84 ± 0.5 |
| PyMT mice treated with | 361 ± 89.3* | 1.07 ± 0.2* |
| Chemotherapy (n = 10) | | |
| PyMT mice treated with the radio- | 195.2 ± 68.8*^{#} | 0.63 ± 0.09*^{#} |
| controlled mRNA (n = 10) | | |

*p<0.05 vs. Control; #p<0.05 vs. Treatment with Chemotherapy

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of irradicate specific cells by a targeting method comprising: administering a mRNA sequence that alter/activate the apoptotic process of the specific cells upon specific stimulation by radio-control external factors; the mRNA sequence includes a radio-control inducible promotor that enables the apoptotic genes to be altered/activated.

2. The method of claim 1, wherein the mRNA sequence is incorporated in lentiviral vector or lipid nanoparticles.

3. The method of claim 1, wherein the mRNA sequence is directed to a polypeptide for P53, Casp 3, Casp 9.

4. The method of claim 1, wherein the mRNA sequence matches the target gene sequence for cancer, Atherosclerosis, diabetes, fatty liver disease and cirrhosis, and other diseases.

5. The method of claim 1, wherein the mRNA sequence is administrated by Local injection or Systemically through an IV intervention.

6. The method of claim 1, further comprising treating target tissues/cells that are tissue specific, cell specific or a combination of both, and localizing the treatment or providing a systemic treatment a body wide treatment.

7. The method of claim 1, wherein the radio-controlled promoter comprises external beam radiotherapy applied to the pelvis over a time period to reverse rectal prolapse, rectal bleeding, and stopping diarrhea associated with colorectal cancer.

8. The method of claim 1, further comprising decreasing the polyp number and size upon treatment with radio-controlled mRNA therapy for colorectal cancer or breast cancer.

9. The method of claim 1, increasing the life expectancy of a subject with colorectal cancer.

10. The method of claim 1, further comprising decreasing the tumor size of breast cancer upon treatment with the radio-controlled mRNA therapy.

11. An in vitro transcription and the construct comprising: an mRNA sequence including a Radio-control inducible promotor that enables the apoptotic genes to be altered/activated; and a specific and personalized coding sequence that matches the target gene sequence in specific cells that are altered in pathophysiological disease.

12. The construct in claim 11, wherein the target sequence targets BRACA1, APC, APOE, GCK, TCF7L2, ABCC8.

13. The construct in claim 11, wherein the mRNA sequence is incorporated in lentiviral vector or lipid nanoparticles.

14. The construct in claim 11, wherein the mRNA sequence is directed to a polypeptide for P53, Casp 3, Casp 9.

15. The construct in claim 11, wherein the mRNA sequence matches the target gene sequence for cancer, Atherosclerosis, diabetes, fatty liver disease and cirrhosis, and other diseases.

16. The construct in claim 11, wherein the mRNA sequence is administrated by Local injection or Systemically through an IV intervention.

17. The construct in claim 11, wherein construct treats target tissues/cells that are tissue specific, cell specific or a combination of both, and provides a systemic treatment a body wide treatment.

18. The construct in claim 11, wherein the radio-controlled promoter comprises external beam radiotherapy applied to the pelvis over a time period to reverse rectal prolapse, rectal bleeding, and stopping diarrhea associated with colorectal cancer.

19. The construct in claim 11, wherein construct decreases the polyp number and size upon treatment with radio-controlled mRNA therapy for colorectal cancer or breast cancer.

20. The construct in claim 11, wherein construct increases the life expectancy of a subject with colorectal cancer.
